(19) United States
(12) Patent Application Publication
(10) Pub. No.: US 2014/0366486 A1
(43) Pub. Date: Dec. 18, 2014

(54) GLOVE BAG MANIFOLD FOR ASEPTIC ASSEMBLING OF RADIOPHARMACEUTICAL FILLING UNITS

(71) Applicant: PIRAMAL IMAGING SA, Matran (CH)

(72) Inventors: Dirk Hinz, Berlin (DE); Christina Hultsch, Berlin (DE); Peter Weinig, Berlin (DE)

(73) Assignee: PIRAMAL IMAGING SA, Matran (CH)

(21) Appl. No.: 14/366,016

(22) PCT Filed: Dec. 20, 2012
(86) PCT No.: PCT/EP2012/076510

§ 371 Date: Jun. 17, 2014

(30) Foreign Application Priority Data
Dec. 21, 2011 (EP) 11194981.4
Feb. 28, 2012 (EP) 12157285.3

(51) Int. Cl. A61B 19/02; A61M 5/178

(52) U.S. Cl. CPC A61B 19/026; A61M 5/1782; A61B 2019/0267
USPC 53/428; 53/510

(57) ABSTRACT

This invention relates to the filling or dispensing of radiopharmaceuticals in sample vials. Radiopharmaceuticals must be dispensed in a cleanroom environment according to cGMP regulations. Single-use filling cassette-type dispensing systems can be used for filling or dispensing of radiopharmaceuticals in sample vials. A single-use filling cassette is the core of such a dispenser system. A flexible bag container comprising a flexible bag and a filling cassette was found. The filling cassette is assembled within the flexible bag under aseptic conditions. The assembled filling cassette defined a closed system since each opening is connected to a sample vials or sterile filter avoiding contamination from outside.

### FIELD OF INVENTION

This invention relates to the filling or dispensing of radiopharmaceuticals in sample vials. Radiopharmaceuticals must be dispensed in a cleanroom environment according to cGMP regulations due to the nature of the injectable solution. No cleanroom for aseptic handling is required when a closed dispensing system is used. Closed systems can be cassette-type-systems with preassemble components; all junctions to the outside are closed via sterile filters. Single-use filling cassette-type dispensing systems can be used for filling or dispensing of radiopharmaceuticals in sample vials. A single-use filling cassette is the core of such a dispenser system. However, the assembling of the closed system needs to be done at the manufacturing site and requires aseptic conditions. These conditions can be realized at least by laminar flow workstations. However, due to the expensiveness and the complexity in maintenance of these workstations this approach can not be implemented at all manufacturing sites. Therefore, a flexible bag container comprising a flexible bag and a filling cassette was found. Said filling cassette is assembled within the flexible bag under aseptic conditions. The assembled filling cassette defined a closed system since each opening is connected to sample vials or sterile filters avoiding contamination from outside. After assembling of the filling cassette under aseptic conditions, the ready filling cassette is mounted onto the single-use filling cassette-type dispensing system.

### BACKGROUND

Radiopharmaceuticals are radioactive pharmaceuticals which are used in nuclear medicine. Radiopharmaceuticals can be used for diagnostics (e.g. [¹⁸F]FDG, [⁹⁹mTc]MIBI) as well as for therapeutic applications (e.g. Zevalin® or [¹³¹I]Iodide). Radiopharmaceuticals are mainly applied in form of injection solutions (e.g. [¹⁸F]FDG, [⁹⁹mTc]MIBI, Zevalin®). Therefore, sterility of the solution needs to be guaranteed. For non-radioactive pharmaceuticals this is often done be terminal sterilization (e.g. heat sterilization). Due to the short half-life of the radiopharmaceutical terminal sterilization can not be performed in many cases. Therefore, radiopharmaceuticals are usually produced, sterilized by filtration and filled under cleanroom conditions.

The manufacture of these radiopharmaceuticals for injection consists of the radiosynthesis including purification and formulation and the filling of the final radiopharmaceutical. During filling, the final solution is diluted if necessary and portioned in adequate sterile vials (e.g. retention vial, quality control vial, vial for sterility testing, and patient vial). Due to the radioactive nature of the product an automated filling process is required. Different filling systems have been described. One possibility is the filling via a robotic arm into open vials and subsequent closing of the vials. A further, often used method is the filling via a closed dispensing system. The transport of the radiopharmaceuticals within the dispensing system is implemented via sterile, single-use tubing and valves. The vials which shall be filled are closed by septa and are attached to the tubings via sterile needles. Ventilation of the vials is performed via needles with sterile filters attached.

According to cGMP guidelines the filling process needs to be performed under aseptic conditions (EudraLex Vol. 4—Annex 1 & 3, 21 CFR Part 212, USP <823>). The whole filling process as well as the closing of the vials needs to be performed under grade A (class 100) conditions. When dispensing is performed via a completely closed system, the filling itself does not need to be performed under class A conditions. However, the assembling of the closed system requires aseptic conditions. These conditions are currently realized by using laminar work benches or isolators.

However, these apparatus are expensive to purchase, they are space-consuming, need to be maintained and its function needs to be controlled continuously.

To circumvent the need of laminar work benches or isolators, a system using a γ-sterilized preassembled flexible bag container for final assembling of the closed dispensing unit was invented.

Single use isolators have been described for the handling of chemicals (e.g. AtmosBag, Sigma Aldrich, U.S. Pat. No. 5,928,075) in order to protect the operator or the chemical from contamination. Furthermore, single use isolators are applied for surgery to establish a sterile field around a surgical instrument (US20110088702, WO2009138813) or the operation site (US20090241970).

Moreover, the aseptic transfer of sterile solutions within a single use isolator was described (EP0812177). However, such a filling concept can not be used for radiopharmaceuticals due to radioprotection reasons.

In addition, systems for aseptic handling and transport of sterile components have been described (EP646868). However, the system disclosed is very complex and rigid and is therefore not applicable for the assembling of the filling unit.

### Problem to be Solved by the Invention and its Solution

According to cGMP guidelines (EudraLex Vol. 4—Annex 3, 21 CFR Part 212, USP <823>) the filling or dispensing of radiopharmaceuticals requires cleanroom conditions. That means radiopharmacies need to have or to establish suitable, qualified labs or compartments. If there is not such a lab, it is possible to use closed systems for the filling processes. Closed systems can be cassette-type-systems with preassemble components; all junctions to the outside are closed via sterile filters. The preassembling of theses cassettes need to be done at the site because permanent piercing of the vials will lead to disruption of the septa and the system is not closed anymore. For assembling of the system a laminar-flow hood (LFH) classified as grade A (class 100) is required to make sure that there is no contamination during the piercing of the septa. In a GMP environment the LFH needs to be qualified and the air quality needs to be checked continuously. For facilities that do not have grade A (class 100) environment for filling and that do not have a qualified LFH the invented single-use-isolator system provides the possibility to perform the assembling of the filling cassette in accordance with cGMP. With the device described in this invention an aseptic preparation of the cassette used for aseptic dispensing of pharmaceuticals could be performed in any surroundings. In general for assembling of a cassette ready to use as closed system cleanroom equipment and procedures as isolator, laminar flow workbench, cleanroom, qualification of equipment, testing for viable and nonviable particles and sanitization is needed. With the present invention nothing of this is needed anymore and there is less spending due to saving of equipment and time for maintenance and requalifications. Therefore also companies or academic sites without cleanroom equipment for aseptic handling could perform dispensing of a sterile pharmaceutical.

### SUMMARY OF THE INVENTION

The pivotal component of the device described in this invention is a single-use isolator or flexible bag made from plastics. This flexible bag container contains all cassette components (e.g. valves, tubings, vials, sterile filters, needles and ventilation needles). Optionally, the cassette components are shrink-wrapped in a further plastic foil or film (primary package). If required, a safety cutter is added in order to open this primary package. A tube with a sterile filter connected is introduced in a turning outward part of the flexible bag. The tube is connected with the flexible bag. The flattened single-use flexible bag is closed, e.g. by shrink-wrapping. At this, a closed single-use flexible bag is obtained. The closed flexible bag is gamma-sterilized. This sterile, closed flexible bag containing all the components of the dispensing cassette can be sent to the manufacturing sites.

At the manufacturing site, the turning outward part of the flexible bag is cut to have access to the tube with the sterile filter. Via the tube, the flexible bag is inflated with sterile gas, e.g. nitrogen or air. Glove recessed portion (glove) integrated into the flexible bag are used to manipulate the components inside the flexible bag. After having assembled the filling cassette, the flexible bag is opened and the filling cassette is connected to the dispensing system. No LFH is required for these steps.

### FIGURES

FIG. 1: Flexible glove bag

Reference: 1: glove location, 2: glove bag, 3: filling cassette, 4: air transfer tube, 5: safety cutter, 6: sample vials, and 8: primary package

FIG. 2: Flexible glove bag with hands.

Reference: 1: glove, 2: glove bag, 3: filling cassette, 4: air transfer tube, 5: safety cutter, 6: sample vials, and 8: primary package.

FIG. 3: Flexible glove bag with hands.

Reference: 1: glove, 2: glove bag, 3: filling cassette, 4: air transfer tube, 5: safety cutter, 6: sample vials, 7: sterile filter and 8: primary package.

### DETAILED DESCRIPTION OF THE INVENTION

In a first aspect, this invention is directed to a flexible bag container for assembling of a filling cassette under aseptic condition wherein the flexible bag container comprises
a flexible bag,
at least one filling cassette and
at least one sample vial.

The assembled filling cassette is then mounted to a filling or dispensing system device and is connected to the bulk solution containing the radiopharmaceutical to be filled in the sample vial(s).

### Flexible Bag:

The flexible bag is comprising at least two opposed walls sealed to each other defining a variable volume chamber with an opening wherein the walls are optically transparent and defining a closed interior. Preferably, the flexible bag is comprising 2 walls sealed to each other with an opening. The opening is closed by a clip, cord or by shrink-wrapping. More preferably, the flexible bag is closed by shrink-wrapping.

The flexible bag comprises additionally at least one infolding to facilitate the assembling of the filling cassette. Preferably, the infoldings are shaped as glove recessed portion (glove) coupled integrally to and extending into the flexible bag allowing from the outside the handling of item(s) present within the flexible bag. The flexible bag has 1 to 4 glove-shaped recessed portions. Preferably the flexible bag has 2 glove-shaped recessed portions.

The flexible bag with the glove-shaped recessed portion (glove) is a glove bag. The flexible bag or glove bag is made of optically transparent plastic material. Preferably, the flexible bag or glove bag is made of plastic material selected from polyethylene, polypropylene, polyphenylene sulfide, polyvinyl chloride, polysulfone, polyethylene terephthalate, ethylen-tetrafluorethylen and fluoroethylene-propylene.

More preferably, the glove bag is made of polyethylene or polypropylene.

The size of the glove bag must be big enough to accommodate the filling cassette and to allow handling of the filling cassette. In a further sub-embodiment, the glove bag is a plastic bag of any shape and size.

Preferably, the flexible bag or the bag glove is of the size of 20 cm×20 cm to 200 cm×200 cm. More preferably, the flexible bag or the bag glove is of the size of 60 cm×70 cm. Preferably, the flexible bag or the bag glove is of the size having a diameter of 20 cm to 200 cm. More preferably, the flexible bag or the bag glove is of the size having a diameter of 40 cm.

Preferably, the glove bag has a round or elliptic as well as a rectangular shape with integrated gloves. More preferably, the glove bag has a rectangular shape.

The glove bag comprises additionally an air transfer tube defining a passage for inflow (inflating) and outflow (compressing) of air or gas to or from the glove bag. The air transfer tube has optionally a closure system limiting entry of air, gas or any liquid into the glove bag. The air transfer tube is comprising a tubing for inflow and outflow of air or gas and sterile filter mounted to the tubing. Preferably, the sterile filter is mounted at one ending of the tubing located within the glove bag or outside the glove bag. More preferably, there is a sterile filter at each ending of the tube.

Sterile gas is air, argon or nitrogen.

Preferably, gas is argon or nitrogen.

The sterile filter is used to prevent any contamination of the inner bag compartment with microorganisms or particles. During inflating of the bag with gas or air, the sterile filter removes potential particles or microorganisms.

Typically, the pore size of the sterile filter is ≦0.22 μm. Sterile filters that are used for this purpose are e.g. Sartorius Midisart 2000 0.2 μm; Whatman Puradisc 30 Syringe Filter or Millipore Millex-GV filters.

The passage of the tube through the glove bag is sealed by shrink-wrapped or the tubing is tied into the glove bag.

As an alternative, the sterile filter is shrink-wrapping the into the glove bag wall defining a passage for inflow (inflating) and outflow (compressing) of air or gas to or from the glove bag.

### Filling Cassette:

The filling cassette is used for preparing single portions of a bulk solution wherein the bulk solution is preferably a radiopharmaceutical containing solution. The filling cassette is fulfilling radiation protection and/or GMP regulation requirements. The filling cassette is used to transfer the radiopharmaceutical containing solution from the bulk vial to the sample vials under aseptic condition.

The filling cassette comprises a system of tubings and valves. The valves are operated by different mechanisms such as pinch valves or actuators. In the flexible bag or glove bag, the filling cassette is connected to the sample vials (e.g. patient vial, QC vial, vial for sterility control, retention vial) via the cassette tubings and the valves. The valves are optionally connected to each other by channels in form of a manifold.

The filling cassette is in a pre-assembled form or assembled form in the flexible bag or glove bag.

Pre-assembled form means that the filling cassette is not connected to the sample vials (e.g. patient vial, QC vial, vial for sterility control, retention vial) via the cassette tubings and the valves.

Assembled form means that the filling cassette is connected to the sample vials (e.g. patient vial, QC vial, vial for sterility control, retention vial) via the cassette tubings and the valves. Connection with the bulk vial occurs after assembling of the filling cassette and removing of the assembled filling cassette from the flexible bag or glove bag. The fluidic pathway is controlled by switching the valves. When the closed filling cassette is mounted to the Single-use filling cassette-type dispensing system and the bulk vial is connected to the assembled closed filling cassette then the radiopharmaceuticals containing solution is transported from the bulk vial through the tubings and the valves and then distributed to the sample vial(s). The transport itself is performed via a syringe that is also connected to one tubing or via gas pressure on the bulk vial or evacuation of the sample vials.

Optionally, the filling cassette comprises filtration unit (filters) for sterile filtration during the filling step.

Preferably, the filling cassette is designated for single-use.

Preferably, the filling cassette consists of materials that are suitable for gamma-sterilization. More preferably, the filling cassette is substantially made of material selected from PP, PE, Polyester, Polysulfone, Polycarbonate, and Polyurethane.

Cassette dispensing systems for the filling of radiopharmaceuticals are e.g. Scintomics InViala, Eckert&Ziegler Modular-Lab PharmTracer or BioScan ReFORM-Plus.

The filling cassette can be packed in a protecting plastic foil or film (primary package). The plastic foil or film packaging has a peel-off-mechanism for opening or is opened with a safety cutter.

The filling cassette can be also packed directly into the flexible bag container without any additional packaging.

Preferably, the flexible bag container comprises 1 to 10 filling cassettes. More preferably, the flexible bag container comprises 1, 2, 3, 4 or 5 filling cassettes. Even more preferably, the flexible bag container comprises 1, 2 or 3 filling cassettes or a single filling cassette.

As shown in the drawings, the flexible bag comprises a single filling cassette optionally packed in a protecting plastic foil or film (primary package).

Radiopharmaceuticals are pharmaceuticals that contain a radioactive isotope. They are used for diagnoses as well as for therapeutic purposes. The radioisotope is an alpha-, a beta- or a gamma-emitter.

Radiopharmaceuticals for diagnostic purposes are e.g. [¹⁸F]FDG, [¹⁸F]FET, [¹⁸F]FLT, [¹⁸F]FMISO, [¹⁸F]FAZA, [¹⁸F]Galacto-RGD, [¹⁸F]FDOPA, [¹⁸F]Flumazenil, [¹⁸F] Annexin, [¹⁸F]Fluorethylcholine, [¹⁸F]Fluormethylcholine, [¹¹C]Methionin, [¹¹]Choline, [¹¹C] Acetate, (2S,4S)-2-Amino-4-(3-[¹⁸F]-fluoropropyl)-pentane dioic acid, (2S)-2-Amino-4-[¹⁸F]-fluoro pentane dioic acid, N-[2-(2-[¹⁸F]Fluoroethoxy)-5-methoxybenzyl]-N-(5-fluoro-2-phenoxyphenyl)acetamide, N-{2-[2-[¹⁸F]fluoroethoxy]-5-methoxybenzyl}-N-[2-(4-methoxyphenoxy)pyridin-3-yl]acetamide, (2RS,4S)-2-[¹⁸F]Fluoro-4-phosphonomethyl-pentanedioic acid, [¹⁸F]Sigma2, [¹⁸F]Bombesin, [⁶⁸Ga]Bombesin, N,N-Diethyl-2-{2-[4-(2-fluoro-ethoxy)-phenyl]-5,7-dimethylpyrazolo[1,5-a]pyrimidin-3-yl}-acetamide, Florbetaben, Florbetapir, Flormetamol, (R)-2-Amino-3-(4-[¹⁸F]fluoromethoxy-phenyl)-propionic acid, [^{99m}Tc]MIBI, [^{99m}Tc]MDP, [^{99m}Tc]DMSA, [^{99m}Tc]HMDP, [^{99m}Tc]HEDP, [^{99m}Tc]HMPAO, [^{99m}Tc]nanocolloides, [^{99m}Tc]macroaggregates, [^{99m}Tc]MAG₃, [^{99m}Tc]ECD, [^{99m}Tc]gluconate, [^{99m}Tc]HIDA, [⁶⁸Ga]DOTATOC, [⁶⁸Ga]DOTANOC, [⁶⁸Ga]DOTATATE.

Radiopharmaceuticals for therapeutic purposes are e.g. [¹⁷⁷Lu]DOTATOC, [¹⁷⁷Lu]DOTATATE, [⁹⁰Y]DOTATOC, [⁹⁰Y]DOTATATE, Zevalin, ²²³Radium chloride. The radiopharmaceuticals mentioned above are solutions for injection.

### Filling Sample Vial:

The filling sample vial is a washed and sterilized container suitable for being filled up with radiopharmaceuticals. Preferably, 1 to 20 filling sample vials are present. More preferably, 1 to 10 filling sample vials are present for each filling cassette. Even more preferably, 1, 2, 3, 4, 5, or 6 filling sample vials are present. Even more preferably, 4 filling sample vials are present. The volume of the flexible bag is depending to the size of the filling cassette, the number of filling sample vials and other items present in the flexible bag.

### Other Items:

The flexible bag container comprises additionally items useful for assembling the filling cassette e.g. ventilation needles and/or a safety cutter for removing the protecting plastic foil or film (primary package) of the filling cassette if there is no peel-off-mechanism.

### Flexible Bag Container:

Flexible bag container comprising a flexible bag, at least one filling cassette and at least one filling sample vial is preferably stored and transported in a flattened (compressed) form or inflated (dilated) form. Flexible bag container is more preferably stored and transported in a compressed form because the risk of a puncture of the flexible bag is reduced. The novel flexible bag container is for single-use purpose.

In a further sub embodiment, the invention is directed to a flexible bag container (8) for assembling of a filling cassette (3) under aseptic condition wherein the flexible bag container (8) comprises
a glove bag (2),
at least one filling cassette (3) and
at least one filling sample vial (6).

Preferably, the flexible bag container for assembling of a filling cassette under aseptic condition comprises
a glove bag with an air transfer tube defining a passage for inflow and outflow of air or gas,
filling cassette(s) wherein filling cassette is optionally packaged in a foil or film and
filling sample vial(s),
   wherein the flexible bag container is in a compressed (flattened) form during storage and transport.

More preferably, the flexible bag container (8) for assembling of a filling cassette (3) under aseptic condition comprises
a glove bag (2) with an air transfer tube (4) defining a passage for inflow and outflow of air or gas wherein the air transfer tube (4) comprises a sterile filter (7),
filling cassette(s) (3) wherein filling cassette (3) is optionally packaged in a foil or film,
filling sample vial(s) (6) and
optionally a safety cutter (5),
   wherein the flexible bag container (8) is in a compressed (flattened) form during storage and transport.

Even more preferably, the flexible bag container for assembling of a filling cassette under aseptic condition comprises
a glove bag with an air transfer tube defining a passage for inflow and outflow of air or gas wherein the air transfer tube comprises a sterile filter,
filling cassette(s) wherein filling cassette is optionally packaged in a foil or film,
filling sample vial(s),
ventilation needles and
optionally a safety cutter,
   wherein the flexible bag container is in a compressed (flattened) form during storage and transport and is gamma-sterilized.

The second aspect of the present invention is directed to a method for obtaining a flexible bag container for assembling of a filling cassette under aseptic condition wherein the flexible bag container comprises
a flexible bag,
at least one filling cassette and
at least one filling sample vial
   comprising the steps of
Introducing filling cassette and sample vial into the flexible bag through the flexible bag opening, and
Closing of the flexible bag

Optionally, the flexible bag is compressed by removing the enclosed air or gas before or after closing the flexible bag step. The bag is closed or it is closed in a way that there is an exchange of gas or air through the air transfer tube. In the last case, the connected sterile filter will prevent any contamination of the inner bag compartment with particles or microorganisms. Air or gas is totally or partially removed from the flexible bag or the glove bag.

Additionally, the flexible bag is sterilized by gamma radiation after the “closing of the flexible bag” step.

Preferably, the “closing of the flexible bag” step is completed by shrink-wrapping.

In a further preferred embodiment, the filling cassette components are first packed in an additional plastic foil or film and are then shrink-wrapped into the glove bag and gamma-sterilized.

In a further preferred embodiment, air is removed from the glove bag prior to the gamma-radiation for sterilization.

In a sub-embodiment, the present invention is directed to a method for obtaining a flexible bag container for assembling of a filling cassette under aseptic condition wherein the flexible bag container comprises
a glove bag,
at least one filling cassette and
at least one filling sample vial
   comprising the steps of
Introducing filling cassette and sample vial into the flexible bag through the flexible bag opening, and
Closing of the flexible bag.

Preferred features and sub-embodiment disclosed in the first aspect are included herein.

The third aspect of the present invention is directed to a method for assembling of a filling cassette under aseptic condition wherein the flexible bag container comprises
a flexible bag,
at least one filling cassette and
at least one filling sample vial
   comprising the step of
Assembling the filling cassette by connecting sample vial to filling cassette.

Additionally, the following steps are occurring before the step “Assembling”
Inflating the flexible bag by introducing sterile air or gas into the flexible bag through an air transfer tube,
Optionally opening and removing the packaging foil or film protecting the filling cassette, and/or
Introducing hand(s) into infolding(s).

Additionally, the following step is occurring after the step “Assembling”
Opening and removing the flexible bag.

Additionally, the following step is occurring after the step “Opening and removing the flexible bag”
Opening and removing of the flexible bag,
Transferring the filling cassette onto the cassette dispensing system
Connecting the filling cassette to the cassette dispensing system and/or to the bulk vial,
Dispensing/filling of the radiopharmaceutical into the sample vials using the filling cassette mounted onto the dispensing cassette system.

Additionally, the following step is occurring after the step “Opening and removing the flexible bag”
Transferring the filling cassette onto the cassette dispensing system
Connecting the filling cassette to the cassette dispensing system and/or to the bulk vial,
Dispensing/filling of the radiopharmaceutical into the sample vials using the filling cassette mounted onto the dispensing cassette system.

Sterile gas is air, argon or nitrogen.

Preferably, gas is argon or nitrogen.

In a preferred embodiment, air or nitrogen is introduced into the glove bag via an integrated sterile filter.

In a sub-embodiment, the present invention is directed to a method for assembling of a filling cassette under aseptic condition wherein the flexible bag container comprises
a glove bag,
at least one filling cassette and
at least one filling sample vial
   comprising the step of
Assembling the filling cassette by connecting sample vial to filling cassette.

Preferred features and sub-embodiment disclosed in the first aspect are included herein.

Filling cassette and filling sample vial are to be understood as one or more Filling cassette and filling sample vial. Preferably, it shall be understood as one filling cassette and/or an adequate set of filling sample vials.

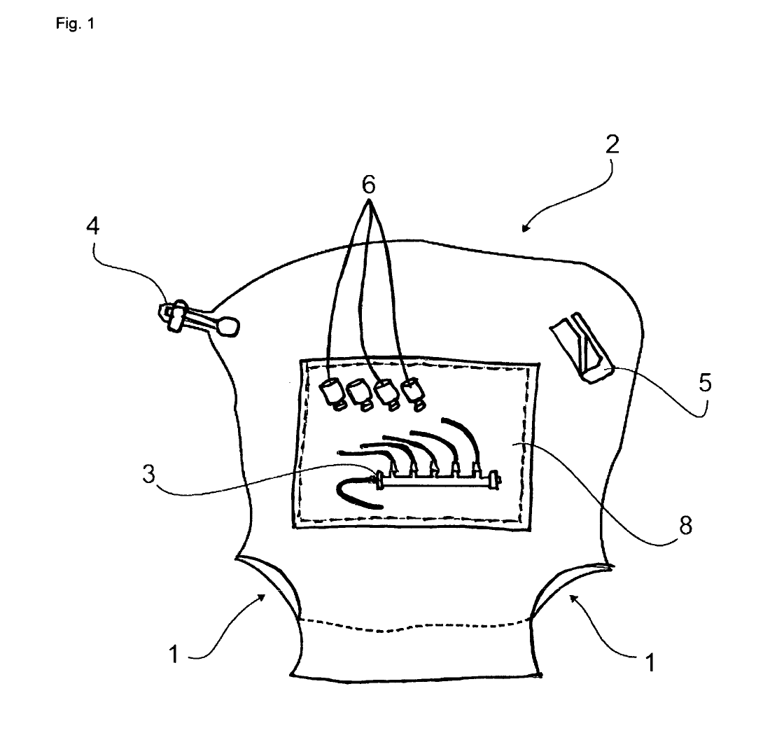

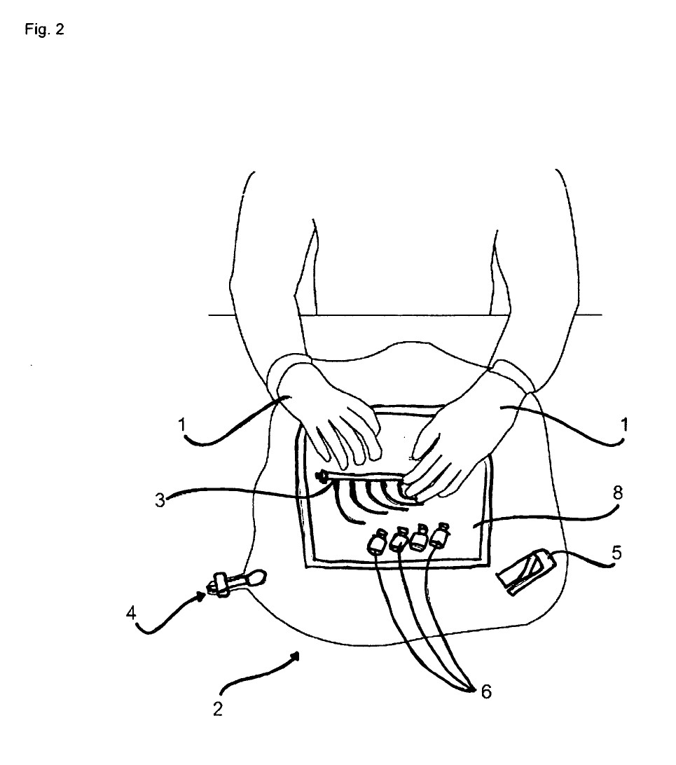

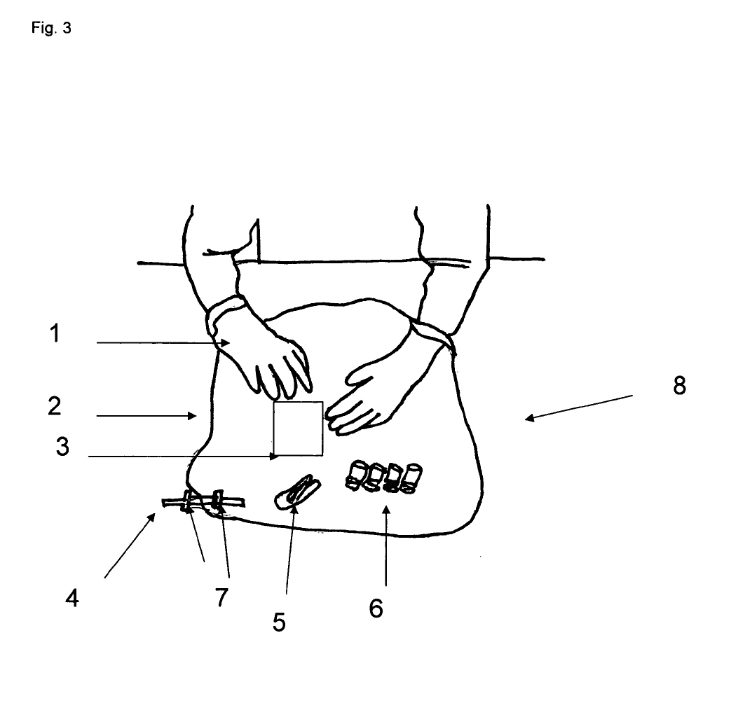

It's not intended to limit invention to feature exemplified in FIGS. 1 to 3.

### Experimental Section

Abbreviations

| | |
|---|---|
| PE | Polyethylene |
| PP | Polypropylene |
| cGMP | Current Good Manufacturing Practice |
| QC | Quality Control |

### General

### Experimental

The described concept was used for the filling of (2RS,4S)-2-[¹⁸F]Fluoro-4-phosphonomethyl-pentanedioic acid. The filling was performed for four independent (2RS,4S)-2-[¹⁸F]Fluoro-4-phosphonomethyl-pentanedioic acid batches. (2RS,4S)-2-[¹⁸F]Fluoro-4-phosphonomethyl-pentanedioic acid was produced in a two-step-two-pot synthesis including [¹⁸F]fluorination, deprotection as well as HPLC and cartridge purifications.

The final (2RS,4S)-2-[¹⁸F]Fluoro-4-phosphonomethyl-pentanedioic acid comprising a volume of 10 ml was filled into a bulk vial.

The glove bag used had dimensions of about 60 cm×70 cm. Two gloves were integrated into the bag. A tube with two sterile filters was attached to the bag. The bag was delivered and stored in compressed form. The bag contained a filling cassette which was shrink-wrapped into another foil. The filling cassette consisted of 6 valves. A needle or a tubing carrying a needle was connected to four valves. A tubing carrying a syringe (20 ml) was connected to on valve. The syringe was used for the liquid transport. A sterile filter was connected to one valve to introduce sterile air for flushing of the filling cassette. A further sterile filter was attached to one valve for sterile filtration of the bulk solution. The sterile filter was attached to the filling cassette with its outlet. A tube with a needle was connected to the sterile filter inlet. Furthermore, 4 ventilation needles with integrated sterile filter and sterile, closed vials (with septa) were placed next to the filling cassette

At first, the glove bag was inflated by introducing argon into the bag using the tube with the sterile filters. This took 5-10 min depending on the gas pressure. After inflating the glove bag, the safety cutter was used to open the foil covering the filling cassette. Then the caps were removed from the needles and from the septa of the vials. The vials were connected to the needles at the filling cassette. Furthermore, each vial was equipped with a ventilation needle. The filling cassette was then a closed system. The glove bag was finally opened by tearing or cutting.

The cassette was attached to the filling unit. The filling unit consisted of valve actuators and a drive mechanism for the syringe.

The needle attached to the sterile filter inlet was inserted into the bulk vial. Furthermore, the bulk vial was equipped with a ventilation needle.

The valves were switched to have a passage (via tubings and channels) from the syringe to the bulk vial. The syringe was then drawn up to a volume of 15 ml. At this the solution was transferred completely from the bulk vial to the syringe. The valves were switched to create a passage to the first vial. 1 ml of solution was dispensed into the first vial (QC vial). The valve were switched again and 6 ml were dispensed into a second vial (patient vial). The valves were switched again and the remaining 3 ml were dosed into a third vial (sterility test). Air was drawn into the syringe via the sterile filter. The air was used for complete transfer of the liquid in the tubings into the vials.

The QC vial was used to determined parameters such as radiochemical purity, chemical purity or radioactivity concentration.

The third vial was used for sterility testing. The testing resulted for all four batches in the outcome “sterile”.

Therefore, the described system fulfilled the requirement to have sterile solutions after filling.

1. A flexible bag container (8) for assembling of a filling cassette (3) under aseptic condition wherein the flexible bag container (8) comprises
a flexible bag (2),
at least one filling cassette (3) and
at least one filling sample vial (6).

2. The flexible bag container according to claim 1 wherein the flexible bag is a glove bag.

3. The flexible bag container according to claim 2 comprising
a glove bag with an air transfer tube defining a passage for inflow and outflow of air or gas wherein the air transfer tube comprises a sterile filter,
filling cassette(s) wherein filling cassette is optionally packaged in a foil or film,
filling sample vial(s),
ventilation needles and
optionally a safety cutter,
wherein the flexible bag container is in a compressed (flattened) form and gamma-sterilized.

4. A method for obtaining a flexible bag container for assembling of a filling cassette under aseptic condition a filling wherein the flexible bag container comprises
a flexible bag,
at least one filling cassette and
at least one filling sample vial
comprising the steps of
Introducing filling cassette and sample vial into the flexible bag through the flexible bag opening, and
Closing of the flexible bag.

5. The method according to claim 4 wherein the flexible bag is a glove bag.

6. The method according to claim 4 wherein the flexible bag is compressed by removing the enclosed air before or after closing the flexible bag step.

7. A method for assembling of a filling cassette under aseptic condition wherein the flexible bag container comprises
a flexible bag,
at least one filling cassette and
at least one filling sample vial
comprising the step of
Assembling the filling cassette by connecting sample vial to filling cassette.

8. The method according to claim 7 wherein the flexible bag is a glove bag.

9. The method according to claim 7 wherein before the step “Assembling” the following steps occur
Inflating the flexible bag by introducing sterile air or gas into the flexible bag through an air transfer tube,
Optionally opening and removing the packaging foil or film protecting the filling cassette, and/or
Introducing hand(s) into infolding(s).

10. The method according to claim 7 wherein after the step “Assembling” the following steps occur
Opening and removing of the flexible bag,
Transferring the filling cassette onto the cassette dispensing system
Connecting the filling cassette to the cassette dispensing system and/or to the bulk vial,
Dispensing/filling of the radiopharmaceutical into the sample vials using the filling cassette mounted onto the dispensing cassette system.
